(19) United States
(12) Patent Application Publication
(10) Pub. No.: US 2012/0014907 A1
(43) Pub. Date: Jan. 19, 2012

(54) USE OF SUBSTITUTED CYANOPYRROLIDINES AND COMBINATION PREPARATIONS CONTAINING THEM FOR TREATING HYPERLIPIDEMIA AND ASSOCIATED DISEASES

(76) Inventors: David Grenville Holmes, Binningen (CH); Thomas E. Hughes, Concord, MA (US)

(21) Appl. No.: 13/245,226

(22) Filed: Sep. 26, 2011

Related U.S. Application Data
(63) Continuation of application No. 10/516,633, filed on Jan. 19, 2005.
(60) Provisional application No. 60/385,220, filed on Jun. 3, 2002.

(51) Int. Cl. A61K 31/738; A61K 31/616; A61K 31/555; A61K 31/455; A61P 9/10; A61K 31/40; A61P 9/00; A61P 3/06; A61P 39/06; A61K 31/785; A61K 31/405

(52) U.S. Cl. 424/78.16; 514/59; 514/161; 514/185; 514/355; 514/356; 514/419; 514/423

(57) ABSTRACT

Disclosed are methods and compositions for the treatment of hyperlipidemia and conditions associated therewith, such as CHD, ischemic stroke, restenosis after angioplasty, peripheral vascular disease, intermittent claudication, myocardial infarction (e.g. necrosis and apoptosis), dyslipidemia and post-prandial lipemia. The methods include administration of a therapeutically effective amount of a compound of formula (I), wherein R is substituted adamantly; and N is 0 to 3; in free form or in acid addition salt form, and a pharmaceutically acceptable carrier.

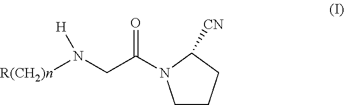

Hyperlipidemia is an important precipitating factor for the premature development of atherosclerosis and increased rate of cardiovascular and peripheral vascular diseases. Hyperlipidemia is a condition generally characterized by an abnormal increase in serum lipids in the bloodstream and is an important risk factor in developing atherosclerosis and heart disease. For a review of disorders of lipid metabolism, see, e.g., Wilson, et al., Ed., Disorders of Lipid Metabolism, Chapter 23, Textbook of Endocrinology, 9^{th} Edition, W.B. Sanders Company, Philadelphia, Pa. (1998); this reference and all references cited therein are herein incorporated by reference. Serum lipoproteins are the carriers for lipids in the circulation and include chylomicrons, very low-density lipoproteins (VLDL), intermediate density lipoproteins (IDL), low density lipoproteins (LDL) and high density lipoproteins (HDL) and lipoprotein a (Lp(a)): Hyperlipidemia is usually classified as primary or secondary hyperlipidemia. Primary hyperlipidemia is generally caused by genetic defects, while secondary hyperlipidemia is generally caused by other factors, such as various disease states, drugs and dietary factors. Alternatively, hyperlipidemia can result from both a combination of primary and secondary causes of hyperlipidemia. Elevated cholesterol levels are associated with a number of disease states, including coronary artery disease, angina pectoris, carotid artery disease, strokes, cerebral arteriosclerosis, and xanthoma.

There are several forms of circulating blood cholesterol which occur naturally in mammals. Some forms are considered “bad” cholesterol, while other forms are considered “good” cholesterol and are essential for good health. The good form of cholesterol has been established to be HDL. LDL is a “bad” cholesterol. Another form of LDL cholesterol, the primary bad form, is Lp(a) which is a modified form of LDL. Elevated levels of Lp(a) are believed to be detrimental and associated with a higher risk for coronary heart disease (CHD) (see Assman et al., Am. J. Card., Vol. 77, pp. 1179-1184 (1996); and Bostom at al., JAMA, Vol. 276, No. 7, pp. 544-548 (1996)). Lowering of Lp(a) levels with a combination of estrogen and progesterone is associated with a lower incidence of detrimental coronary events (see Shlipak et al., JAMA, Vol. 283, No. 14, pp. 1845-1852 (2000)).

Lowering LDL, the bad form of cholesterol, is now one of the primary objectives of physicians treating patients who have or who have a high risk of developing, cardiovascular diseases, such as CHD, atherosclerosis, myocardial infarction, stroke, cerebral infarction, and even restenosis following balloon angioplasty. Many physicians are now utilizing cholesterol-lowering agents purely as a prophylactic treatment in healthy subjects whose cholesterol levels are normal, thereby guarding against development of cardiovascular diseases.

The most commonly used cholesterol-lowering agents are the statins, which are compounds which inhibit the enzyme 3-hydroxy-3-methylglutarylcoenzyme A (HMG-CoA) reductase, the enzyme responsible for catalyzing the conversion of HMG-CoA to mevalonate, which is an early and rate-limiting step in the cholesterol biosynthetic pathway.

Due to these debilitating effects of hyperlipidemia, there is a need for new therapeutic methods and compositions for modulating, treating or preventing hyperlipidemia and conditions associated therewith.

Toward these ends and others, in one aspect the present invention there is provided a method of modulating hyperlipidemia and/or conditions associated with hyperlipidemia comprising administering to a mammal in need thereof a therapeutically effective amount of a compound of formula I:

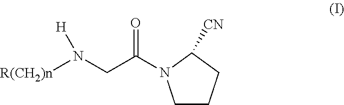

wherein
R is substituted adamantyl; and
N is 0 to 3; in free form or in acid addition salt form.

Furthermore, the present invention relates to the use of a compound of formula I, or a pharmaceutically acceptable salt thereof, for the manufacture of a medicament for modulating hyperlipidemia and/or conditions associated with hyperlipidemia.

The invention furthermore relates to a pharmaceutical composition for modulating hyperlipidemia and/or conditions associated with hyperlipidemia comprising a compound of formula I, or a pharmaceutically acceptable salt thereof.

Preferably a method of modulating hyperlipidemia comprising administering to a mammal in need thereof a therapeutically effective amount of a compound of formula I:

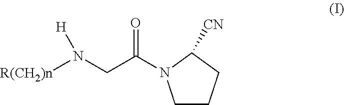

wherein
R is substituted adamantyl; and
N is 0 to 3; in free form or in acid addition salt form.

Preferably, the present invention relates to the use of a compound of formula I, or a pharmaceutically acceptable salt thereof, for the manufacture of a medicament for modulating hyperlipidemia.

Preferably the invention furthermore relates to a pharmaceutical composition for modulating hyperlipidemia comprising a compound of formula I, or a pharmaceutically acceptable salt thereof.

Preferably the present invention relates to the use of a therapeutically effective amount of a compound of formula I, or a pharmaceutically acceptable salt thereof.

Preferred are the compounds of formulae IA or IB:

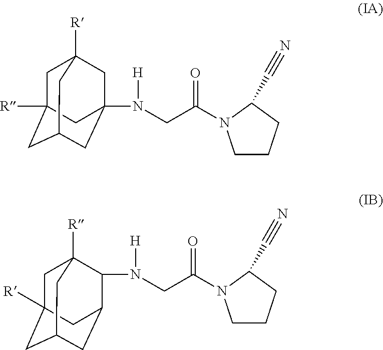

wherein R′ represents hydroxy, C₁-C₇alkoxy, C₁-C₈-alkanoyloxy or R₅ R₄ N—CO—O—, where R₄ and R₅ independently are C₁-C₇alkyl or phenyl which is unsubstituted or substituted by a substituent selected from C₁-C₇alkyl, C₁-C₇alkoxy, halogen and trifluoromethyl and where R₄ additionally is hydrogen; or R₄ and R₅ together represent C₃-C₆alkylene; and R″ represents hydrogen; or R′ and R″ independently represent C₁-C₇alkyl; in free form or in form of a pharmaceutically acceptable acid addition salt.

Most preferred is the compound of formula IC:

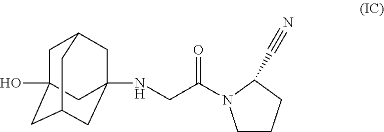

also referred to as pyrrolidine, 1-[3-hydroxy-1-adamantyl)amino]acetyl-2-cyano-, (S) and its pharmaceutically acceptable acid addition salts.

In another preferred aspect the present invention there is provided a method of modulating conditions associated with hyperlipidemia comprising administering to a mammal in need thereof a therapeutically effective amount of a compound of formulae I, IA, IB or IC as described above or pharmaceutically acceptable acid addition salts thereof.

Preferably, the present invention relates to the use of a therapeutically effective amount of a compound of formulae I, IA, IB or IC as described above, or a pharmaceutically acceptable salt thereof, for the manufacture of a medicament for modulating conditions associated with hyperlipidemia.

Preferably the invention furthermore relates to a pharmaceutical composition for modulating conditions associated with hyperlipidemia, comprising a therapeutically effective amount of a compound of formulae I, IA, IB or IC as described above, or a pharmaceutically acceptable salt thereof.

Conditions associated with hyperlipidemia include atherosclerosis, angina pectoris, carotid artery disease, cerebral arteriosclerosis, xanthoma, CHD, ischemic stroke, restenosis after angioplasty, peripheral vascular disease, intermittent claudication, myocardial infarction (e.g. reduction in necrosis), dyslipidemia, post-prandial lipemia.

In another aspect of the present invention there is provided a method of lowering VLDL, LDL and Lp(a) levels in a mammal comprising administering a therapeutically effective amount of a compound of formulae I, IA, IB or IC as described above or pharmaceutically acceptable acid addition salts thereof.

Furthermore, the present invention relates to the use of a therapeutically effective amount of a compound of formulae I, IA, IB or IC as described above, or a pharmaceutically acceptable salt thereof, for the manufacture of a medicament for lowering VLDL, LDL and Lp(a) levels in a mammal.

The invention furthermore relates to a pharmaceutical composition for lowering VLDL, LDL and Lp(a) levels in a mammal, comprising a therapeutically effective amount of a compound of formulae I, IA, IS or IC as described above, or a pharmaceutically acceptable salt thereof.

In another aspect of the present invention there is provided a pharmaceutical composition comprising a therapeutically effective amount of the compound of formulae I, IA, IB or IC and a pharmaceutically acceptable carrier.

Other objects, features, advantages and aspects of the present invention will become apparent to those of skill from the following description, appended claims and accompanying drawings. It should be understood, however, that the following description, appended claims, drawings and the specific examples, while indicating preferred embodiments of the invention, are given by way of illustration only. Various changes and modifications within the spirit and scope of the disclosed invention will become readily apparent to those skilled in the art from reading the following.

Unless otherwise specified herein, common definitions are intended by the words and terms used herein. As throughout this specification the singular is intended to include the plural and vice versa.

The term “therapeutically effective amount” shall mean that amount of compound that will elicit the biological or medical response of a tissue, system or animal (mammal) that is being sought by a researcher or clinician.

The terms “mammal”, “mammalian organism”, “subject” or “patient” are used interchangeably herein and include, but are not limited to, humans, dogs, cats, horses, pigs, cows, monkeys, rabbits, mice and laboratory animals. The preferred mammals are humans.

The term “modulate” refers to the treating, prevention, suppression, enhancement or induction of a function or condition. For example, the compounds of the present invention can modulate hyperlipidemia by lowering cholesterol in a human, thereby suppressing hyperlipidemia.

The term “treating” means the management and care of a human subject for the purpose of combating the disease, condition or disorder and includes the administration of a compound of the present invention to prevent the onset of the symptoms or complications, alleviating the symptoms or complications, or eliminating the disease, condition or disorder.

The term “elevated levels of Lp(a)” as used herein shall mean levels of Lp(a) which subjects the patient to the risk of vascular, particularly cardiovascular diseases, mediated by Lp(a), including but not limited to CHD, ischemic stroke, restenosis after angioplasty, peripheral vascular disease, intermittent claudication, myocardial infarction (e.g. reduction in necrosis), dyslipidemia and post-prandial lipemia.

The term “hyperlipidemia” refers to the presence of an abnormally elevated level of lipids in the blood. Hyperlipidemia can appear in at least three forms: (1) hypercholesterolemia, i.e., an elevated cholesterol level; (2) hypertriglyceridemia, i.e., an elevated triglyceride level; and (3) combined hyperlipidemia, i.e., a combination of hypercholesterolemia and hypertriglyceridemia. This term also refers to elevated levels of one or more lipoproteins, e.g., elevated levels of Lp(a), LDL and/or VLDL.

The term “cholesterol” refers to a steroid alcohol that is an essential component of cell membranes and myelin sheaths and, as used herein, Incorporates its common usage. Cholesterol also serves as a precursor for steroid hormones and bile acids.

The term “triglyceride(s)” (TGs), as used herein, incorporates its common usage. TGs consist of three fatty acid molecules esterified to a glycerol molecule and serve to store fatty acids which are used by muscle cells for energy production or are taken up and stored in adipose tissue.

Because cholesterol and TGs are water insoluble, they must be packaged in special molecular complexes known as “lipoproteins” in order to be transported in the plasma. Lipoproteins can accumulate in the plasma due to overproduction and/or deficient removal. There are at least five distinct lipoproteins differing in size, composition, density and function. In the cells of the small of the intestine, dietary lipids are packaged into large lipoprotein complexes called “chylomicrons”, which have a high TG and low cholesterol content. In the liver, TG and cholesterol esters are packaged and released into plasma as TG-rich lipoprotein called VLDL, whose primary function is the endogenous transport of TGs made in the liver or released by adipose tissue. Through enzymatic action, VLDL can be either reduced and taken up by the liver, or transformed into IDL. IDL, is in turn, either taken up by the liver, or is further modified to form the LDL. LDL is either taken up and broken down by the liver, or is taken up by extrahepatic tissue. HDL helps remove cholesterol from peripheral tissues in a process called reverse cholesterol transport.

Exemplary primary hyperlipidemia include, but are not limited to, the following:

1) Familial hyperchylomicronemia, a rare genetic disorder which causes a deficiency in an enzyme, LP lipase, that breaks down fat molecules. The LP lipase deficiency can cause the accumulation of large quantities of fat or lipoproteins in the blood;
2) Familial hypercholesterolemia, a relatively common genetic disorder caused where the underlying defect is a series of mutations in the LDL receptor gene that result in malfunctioning LDL receptors and/or absence of the LDL receptors. This brings about ineffective clearance of LDL by the LDL receptors resulting in elevated LDL and total cholesterol levels in the plasma;
3) Familial combined hyperlipidemia, also known as multiple lipoprotein-type hyperlipidemia; an inherited disorder where patients and their affected first-degree relatives can at various times manifest high cholesterol and high triglycerides. Levels of HDL cholesterol are often moderately decreased;
4) Familial defective apolipoprotein B-100 is a relatively common autosomal dominant genetic abnormality. The defect is caused by a single nucleotide mutation that produces a substitution of glutamine for arginine which can cause reduced affinity of LDL particles for the LDL receptor. Consequently, this can cause high plasma LDL and total cholesterol levels;
5) Familial dysbetaliproteinemia, also referred to as Type III hyperlipoproteinemia, is an uncommon inherited disorder resulting In moderate to severe elevations of serum TG and cholesterol levels with abnormal apolipoprotein E function. HDL levels are usually normal; and
6) Familial hypertriglyceridemia, is a common inherited disorder in which the concentration of plasma VLDL is elevated. This can cause mild to moderately elevated triglyceride levels (and usually not cholesterol levels) and can often be associated with low plasma HDL levels. Risk factors in exemplary secondary hyperlipidemia include, but are not limited to, the following: (1) disease risk factors, such as a history of Type 1 diabetes, Type 2 diabetes, Cushing's syndrome, hypothyroidism, cholestasis and certain types of renal failure; (2) drug risk factors, which include, birth control pills; hormones, such as estrogen and corticosteroids; certain diuretics; and various β-blockers; (3) dietary risk factors include dietary fat intake per total calories greater than 40%; saturated fat intake per total calories greater than 10%; cholesterol intake greater than 300 mg per day; habitual and excessive alcohol use; bulimia, anorexia nervosa, and obesity.

“Pharmaceutically acceptable salt(s)” refer to the non-toxic alkali metal, alkaline earth metal, and ammonium salts commonly used in the pharmaceutical industry including the sodium, potassium, lithium, calcium, magnesium, barium, ammonium and protamine zinc salts, which are prepared by methods well-known in the art. The term also includes non-toxic acid addition salts, which are generally prepared by reacting the compounds of the present invention with a suitable organic or inorganic acid. Representative salts include, but are not limited to, the hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, napsylate and the like.

“Pharmaceutically acceptable acid addition salt(s)” refers to those salts which retain the biological effectiveness and properties of the free bases and which are not biologically or otherwise undesirable, formed with Inorganic acids, such as hydrochloric acid, hydrobromic acid, sulfuric acid, nitric acid, phosphoric acid and the like; and organic acids, such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, menthanesulfonic acid, ethanesulfonic acid, p-toluenesulfonic acid, salicylic acid and the like. For a description of pharmaceutically acceptable acid addition salts as prodrugs see, e.g., Bundgaard, Ed., Design of Prodrugs, Elsevier Science Publishers, Amsterdam (1985)).

An aspect of the present Invention provides a method of modulating hyperlipidemia comprising administering to a mammal in need thereof a therapeutically effective amount of a compound of formula I:

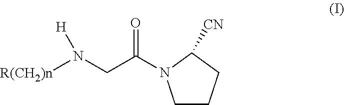

wherein
R is substituted adamantyl; and
N is 0 to 3; in free form or in acid addition salt form.

Preferred are the compounds of formulae IA or IB:

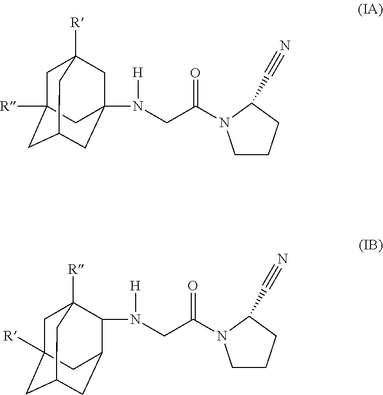

wherein R′ represents hydroxy, C₁-C₇alkoxy, C₁-C₈alkanoyloxy or R₅ R₄ N—CO—O—, where R₄ and R₅ independently are C₁-C₇alkyl or phenyl which is unsubstituted or substituted by a substituent selected from C₁-C₇alkyl, C₁-C₇alkoxy, halogen and trifluoromethyl and where R₄ additionally is hydrogen; or R₄ and R₅ together represent C₃-C₆alkylene; and R″ represents hydrogen; or R′ and R″ independently represent C₁-C₇alkyl; in free form or in form of a pharmaceutically acceptable acid addition salt.

Most preferred is the compound of formula IC:

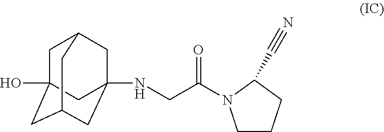

also referred to as pyrrolidine, 1-[3-hydroxy-1-adamantyl)amino]acetyl-2-cyano-, (S) or its pharmaceutically acceptable acid addition salts.

Another aspect of the present invention provides a method of modulating conditions associated with hyperlipidemia comprising administering to a mammal in need thereof a therapeutically effective amount of a compound of formula I:

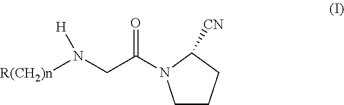

wherein
R is substituted adamantyl; and
N is 0 to 3; in free form or in acid addition salt form.

Preferred are the compounds of formulae IA or IB:

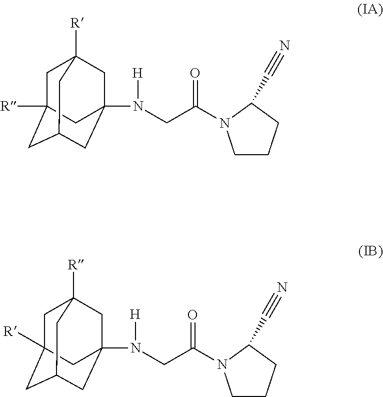

wherein R′ represents hydroxy, C₁-C₇alkoxy, C₁-C₈-alkanoyloxy, or R₅, R₄ N—CO—O—, where R₄ and R₅ independently are C₁-C₇alkyl or phenyl which is unsubstituted or substituted by a substituent selected from C₁-C₇alkyl, C₁-C₇alkoxy, halogen and trifluoromethyl and where R₄ additionally is hydrogen; or R₄ and R₅ together represent C₃-C₆alkylene; and R″ represents hydrogen; or R′ and R″ independently represent C₁-C₇alkyl; in free form or in form of a pharmaceutically acceptable acid addition salt.

Especially preferred is the compound of formula IC:

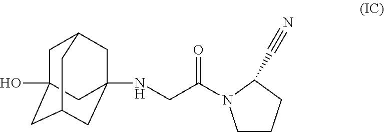

and its pharmaceutically acceptable acid addition salts.

Included with the scope of the present invention are pharmaceutically acceptable salts and pharmaceutically acceptable acid addition salts of the compounds of formulae I, IA, IB and IC.

The compounds of this invention, compounds I, IA, IB and IC, are disclosed in U.S. Pat. No. 6,166,063 issued Dec. 26, 2000 and PCT publication WO 00/34241 published Jun. 15, 2000, the disclosures of which are hereby incorporated by reference herein in their entirety as if set forth in full herein.

Conditions associated with hyperlipidemia include, but are not limited to, atherosclerosis, angina pectoris, carotid artery disease, cerebral arteriosclerosis, xanthoma, CHD, ischemic stroke, restenosis after angioplasty, peripheral vascular disease, intermittent claudication, myocardial infarction, dyslipidemia, post-prandial lipemia.

Another aspect of the present invention relates to lowering levels of Lp(a), LDL and/or VLDL in a mammal comprising administering a therapeutically effective amount of a compound of formulae I, IA, IB or IC to a mammal.

In yet another aspect of the present invention there are provided pharmaceutical compositions comprising a therapeutically effective amount of the compound of formula I or an acid addition salt thereof and a pharmaceutically acceptable carrier. Preferably the compound is (S)-1-(2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine or a pharmaceutically acceptable salt thereof. Preferably the compound is pyrrolidine, 1-[3-hydroxy-1-adamantyl)amino]acetyl-2-cyano, (S).

The compounds of formula I, and their corresponding pharmaceutically acceptable acid addition salts, may be combined with one or more pharmaceutically acceptable carriers and, optionally, one or more other conventional pharmaceutical adjuvants and administered enterally, e.g., orally, in the form of tablets, capsules, caplets, etc. or parenterally, e.g., intravenously, in the form of sterile injectable solutions or suspensions. The enteral and parenteral compositions may be prepared by conventional means.

The compounds of formula I, and their corresponding pharmaceutically acceptable acid addition salts, may be formulated into enteral and parenteral pharmaceutical compositions containing an amount of the active substance that is effective for modulating, treating or preventing hyperlipidemia and conditions associated with hyperlipidemia and for lowering levels of Lp(a), LDL and/or VLDL, in unit dosage form and such compositions comprising a pharmaceutically acceptable carrier.

The compounds of formula I, including those of each of the subscopes thereof and each of the examples, may be administered in enantiomerically pure form, e.g. >98%, preferably >99%; or together with the R enantiomer, e.g., in racemic form. The above dosage ranges are based on the compounds of formula I (excluding the amount of the R enantiomer).

The precise dosage of the compounds of formula I, and their corresponding pharmaceutically acceptable acid addition salts, to be employed for modulating, treating or preventing hyperlipidemia and conditions associated with hyperlipidemia, and for lowering levels of Lp(a), LDL and/or VLDL, depends upon several factors, including the host, the nature and the severity of the condition being treated, the mode of administration and the particular compound employed. However, in general, hyperlipidemia and conditions associated with hyperlipidemia are effectively treated when compounds of formula I, or a corresponding pharmaceutically acceptable acid addition salt, is administered enterally, e.g., orally or parenterally, e.g., Intravenously, preferably orally, at a daily dosage of 0.002-5 mg/kg, preferably 0.02-2.5 mg/kg body weight or, for most larger primates, a daily dosage of 0.1-250 mg/kg, preferably 1-100 mg/kg. A typical oral dosage unit is 0.01-0.75 mg/kg, one to three times a day. Usually, a small dose is administered initially and the dosage is gradually increased until the optimal dosage for the host under treatment is determined. The upper limit of dosage is that imposed by side effects and can be determined by trial for the host being treated.

The compounds of the present invention can be used effectively alone or in combination with one or more additional active agents depending on the desired target therapy. A number of studies have investigated the benefits of combination therapies with oral agents (see, e.g., Mahler, J. Clin. Endocrinol. Metab., Vol. 84, pp. 1165-1171 (1999); United Kingdom Prospective Diabetes Study Group: UKPDS 28, Diabetes Care, Vol. 21, pp. 87-92 (1998); Bardin, Ed., Current Therapy In Endocrinology and Metabolism, 6^{th} Edition, Mosby-Year Book, Inc., St. Louis, Mo. (1997); Chiasson et al., Ann. Intern. Med., Vol. 121, pp. 928-935 (1994); Coniff et al., Clin. Ther., Vol. 19, pp. 16-26 (1997); Coniff et al., Am. J. Med., Vol. 98, pp. 443-451 (1995); Iwamoto et al, Diabet. Med., Vol. 13, pp. 365-370 (1996); and Kwiterovich, Am. J. Cardiol., Vol. 82, No. 12A, pp. 3U-17U (1998)). These studies indicate that diabetes and hyperlipidemia modulation can be further improved by the addition of a second agent to the therapeutic regimen. Combination therapy includes administration of a single pharmaceutical dosage formulation which contains a compound having the general structure of formula I (or formulae IA, IB or IC) and one or more additional active agents, as well as administration of a compound of formula I (or formulae IA, IB or IC) and each active agent in its own separate pharmaceutical dosage formulation. For example, a compound of formula I and an HMG-CoA reductase inhibitor can be administered to the human subject together in a single oral dosage composition, such as a tablet or capsule, or each agent can be administered in separate oral dosage formulations. Where separate dosage formulations are used, a compound of formula I and one or more additional active agents can be administered at essentially the same time, i.e., concurrently; or at separately staggered times, i.e., sequentially. Combination therapy is understood to include all these regimens. Thus the invention furthermore relates to a combination especially a pharmaceutical combination, such as a combined preparation or pharmaceutical composition, respectively, comprising a compound of formula I preferably a compound of formula IA, IB or IC, or a pharmaceutically acceptable salt thereof and at least one active agent selected from the group consisting of an antihyperlipidemic agent; a plasma HDL-raising agent; an antihypercholesterolemic agent, such as a cholesterol biosynthesis inhibitor, e.g., an HMG-CoA reductase inhibitor (also referred to as statins, such as lovastatin, simvastatin, pravastatin, fluvastatin and atorvastatin), an HMG-CoA synthase inhibitor, a squalene epoxidase inhibitor or a squalene synthetase inhibitor (also known as squalene synthase inhibitor); an ACAT inhibitor, such as melinamide; probucol; nicotinic acid and the salts thereof and niacinamide; a cholesterol absorption inhibitor, such as β-sitosterol or ezetimibe; a bile acid sequestrant anion exchange resin, such as cholestyramine, colestipol or dialkylaminoalkyl derivatives of a cross-linked dextran; an LDL receptor inducer; a cholesterol absorption inhibitor such as ezitimibe; fibrates, such as clofibrate, bezafibrate, fenofibrate and gemfibrozil; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins, such as vitamin C and E and β-carotene; a β-blocker; an angiotensin II receptor (AT₁) antagonist; an angiotensin-converting enzyme inhibitor; a renin inhibitor, and a platelet aggregation inhibitor, such as fibrinogen receptor antagonists, i.e., glycoprotein IIb/IIIa fibrinogen receptor antagonists; and aspirin. As noted above, the compounds of formula I can be administered in combination with more than one additional active agent, for example, a combination of a compound of formula I with an HMG-CoA reductase inhibitor, e.g., lovastatin, simvastatin, atorvastatin and pravastatin; and aspirin, or a compound of formula I with an HMG-CoA reductase inhibitor and a β-blocker.

Thus the invention furthermore relates to a combination especially a pharmaceutical combination, which comprises (a) a compound of formula I preferably a compound of formula IA, IB or IC, and at least one compound selected from (b) an antihyperlipidemic agent; a plasma HDL-raising agent; an antihypercholesterolemic agent, such as a cholesterol biosynthesis inhibitor, e.g., an HMG-CoA reductase inhibitor (also referred to as statins, such as lovastatin, simvastatin, pravastatin, fluvastatin and atorvastatin), an HMG-CoA synthase inhibitor, a squalene epoxidase inhibitor or a squalene synthetase inhibitor (also known as squalene synthase Inhibitor); an ACAT inhibitor, such as melinamide; probucol; nicotinic acid and the salts thereof and niacinamide; a cholesterol absorption inhibitor, such as β-sitosterol or ezetimibe; a bile acid sequestrant anion exchange resin, such as cholestyramine, colestipol or dialkylaminoalkyl derivatives of a cross-linked dextran; an LDL receptor inducer; a cholesterol absorption inhibitor such as ezitimibe; fibrates, such as clofibrate, bezafibrate, fenofibrate and gemfibrozil; vitamin, B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins, such as vitamin C and E and β-carotene; a β̅blocker; an angiotensin II receptor (AT₁) antagonist; an angiotensin-converting enzyme inhibitor; a renin inhibitor, and a platelet aggregation inhibitor, such as fibrinogen receptor antagonists, i.e., glycoprotein IIb/IIIa fibrinogen receptor antagonists; and aspirin. As noted above, the compounds of formula I can be administered in combination with more than one additional active agent, for example, a combination of a compound of formula I with an HMG-CoA reductase inhibitor, e.g., lovastatin, simvastatin, atorvastatin and pravastatin; and aspirin, or a compound of formula I with an HMG-CoA reductase inhibitor and a β̅blocker, wherein the active Ingredients are present independently of each other in free form or in the form of a pharmaceutically acceptable salt and optionally at least one pharmaceutically acceptable carrier; for simultaneous, separate or sequential use.

Combination as described above which is a combined, preparation or a pharmaceutical composition.

A pharmaceutical composition comprising a combination which comprises (a) a compound of formula I, preferably a compound of formula IA, IB or IC, and at least one compound selected from the group (b) and wherein the active ingredients are present Independently of each other in free form or in the form of a pharmaceutically acceptable salt and optionally at least one pharmaceutically acceptable carrier.

A pharmaceutical composition comprising a quantity which is jointly therapeutically effective against herein mentioned diseases of a combination which comprises (a) a compound of formula I preferably a compound of formula IA, IB or IC, and at least one compound selected from the group (b)

A pharmaceutical composition comprising a quantity which is jointly therapeutically effective against herein mentioned diseases of a combination as described above and at least one pharmaceutically acceptable carrier.

The term “at least one active agent” shall mean that in addition to the compound of formula (I) one or more, for example two, furthermore three, active ingredients as specified according to the present invention can be combined.

The invention furthermore relates to a method for modulating conditions associated with hyperlipidemia and/or for lowering VLDL, LDL and Lp(a) levels in a mammal, comprising administering to a mammal in need thereof a therapeutically effective amount of a compound of formula I:

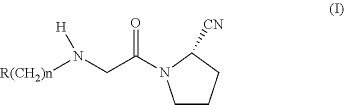

wherein
R is substituted adamantyl;
N is 0 to 3; in free form or in acid addition salt form; and
another active agent.

The invention furthermore relates to the use of a pharmaceutical combination comprising a compound of formula I:

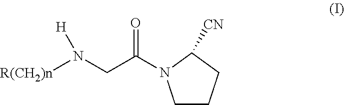

wherein
R is substituted adamantyl;
N is 0 to 3; in free form or in acid addition salt form; and
another active agent, for the manufacture of a medicament for modulating hyperlipidemia, for modulating conditions associated with hyperlipidemia and/or for lowering VLDL, LDL and Lp(a) levels in a mammal.

The invention furthermore relates to the use of a pharmaceutical combination as described herein for the manufacture of a medicament for modulating hyperlipidemia, for modulating conditions associated with hyperlipidemia and/or for lowering VLDL, LDL and Lp(a) levels in a mammal.

The invention furthermore relates to uses or methods of treatment as described herein, wherein the compound of formula I is administered in the form of a pharmaceutical combination or composition as described above.

The invention furthermore relates to a pharmaceutical composition for lowering VLDL, LDL and Lp(a) levels in a mammal, comprising a combination as described herein, or a pharmaceutically acceptable salt thereof.

Preferred compounds of formula I are compounds of formula IA, IB or IC as described herein.

Combination as described above which is a combined, preparation or a pharmaceutical composition.

A pharmaceutical composition as described above comprising a quantity which is jointly therapeutically effective against herein mentioned diseases of a combination as described above and at least one pharmaceutically acceptable carrier.

The structure of the active agents identified by generic or tradenames may be taken from the actual edition of the standard compendium “The Merck Index” or from databases, e.g. patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo. The corresponding active ingredients or a pharmaceutically acceptable salts thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All the more surprising is the experimental finding that the combined administration of formula I or a salt thereof and a therapeutic agent (active agent) selected from the group mentioned below results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of the compound of formula (I) with a therapeutic agent selected from the group described herein results in a more effective prevention or preferably treatment of diseases specified herein. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects, e.g. less increase of weight, on diseases and conditions associated with diabetes mellitus, for a number of combinations as described herein. Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the compounds of formula (I) or of the combination of the active agents used according to the present invention can be demonstrated e.g. by using corresponding pharmacological models known in the pertinent art.

The pharmaceutical composition according to the present invention as described hereinbefore and hereinafter may be used for simultaneous use or sequential use in any order, for separate use or as a fixed combination.

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated. Preferably, the jointly therapeutically effective amounts of the active agents according to the combination of the present invention can be administered simultaneously or sequentially in any order, separately or in a fixed combination.

An example of combination therapy that modulates (prevents the onset of the symptoms or complications associated) atherosclerosis, wherein a compound of formula I is administered in combination with one or more of the following active agents (b): an antihyperlipidemic agent; a plasma HDL-raising agent; an antihypercholesterolemic agent, such as a cholesterol biosynthesis inhibitor, e.g., an HMG-CoA reductase inhibitor (also referred to as statins, such as lovastatin, simvastatin, pravastatin, fluvastatin and atorvastatin), an HMG-CoA synthase inhibitor, a squalene epoxidase inhibitor or a squalene synthetase inhibitor (also known as squalene synthase inhibitor); an acyl-coenzyme A cholesterol acyltransferase (ACAT) inhibitor, such as melinamide; probucol; nicotinic acid and the salts thereof; and niacinamide; a cholesterol absorption inhibitor, such as β-sitosterol or ezetimibe; a bile acid sequestrant anion exchange resin, such as cholestyramine, colestipol, colesevelam or dialkylaminoalkyl derivatives of a cross-linked dextran; an inhibitor of cholesterol absorption, such as ezitimibe; an LDL receptor inducer; fibrates, such as clofibrate, bezafibrate, fenofibrate and gemfibrozil; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide, supra); anti-oxidant vitamins. such as vitamin C and E and β-carotene; a β blocker; an angiotensin II receptor (AT₁) antagonist; an angiotensin-converting enzyme inhibitor, a renin inhibitor; and a platelet aggregation inhibitor, such as fibrinogen receptor antagonists, i.e., glycoprotein IIb/IIIa fibrinogen receptor antagonists; and aspirin. As noted above, the compounds of formula I can be administered in combination with more than one additional active agent, for example, a combination of a compound of formula I with an HMG-CoA reductase inhibitor, e.g., lovastatin, simvastatin, atorvastatin and pravastatin; and aspirin, or a compound of formula I with an HMG-CoA reductase inhibitor and a β-blocker.

A further example of a preferred combination therapy can be seen in modulating hyperlipidemia, wherein the compounds of formula I can be effectively used in combination with, for example, statins, i.e., fluvastatin, lovastatin, pravastatin, atorvastatin or simvastatin; bile acid-binding resins, i.e., colestipol or cholestyramine; nicotinic acid, probucol, β-carotene, vitamin E or vitamin C. Preferably the compound of formula I is a compound of formula IC. Preferably the active agent (b) is selected from the group consisting of fluvastatin, lovastatin, pravastatin, atorvastatin or simvastatin.

A further aspect of the present invention is a kit for the prevention of, delay of progression of, treatment of a disease or condition according to the present invention comprising

(a) an amount of a compound of formula I or a pharmaceutically acceptable salt thereof in a first unit dosage form;
(b) an amount of at least one therapeutic agent selected from the group consisting of components (active agents (b)) as described above, or, in each case, where appropriate, a pharmaceutically acceptable salt thereof in a second etc. unit dosage form; and
(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a compound of formula I or a pharmaceutically acceptable salt thereof in a first unit dosage form;
(b) an amount of at least one therapeutic agent selected from the group consisting of components (active agents) as described above or, in each case, where appropriate, a pharmaceutically acceptable salt thereof, in the form of two or three or more separate units of the components described above.

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components as described above, together with instructions for its simultaneous, separate or sequential use, or any combination thereof, in the delay of progression or treatment of the diseases as mentioned herein. Preferred compounds of formula I are compounds of formula IA, IB or IC as described herein. Preferred active agents (b) are described above

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g.

The doses of compounds of formula (I) to be administered to warm-blooded animals, for example human beings, of, for example, approximately 70 kg body weight, especially the doses effective in the inhibition of the enzyme renin, e.g. In lowering blood pressure and/or in improving the symptoms of glaucoma, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

### EXAMPLES

The present invention is further described by the following example. The example is provided solely to illustrate the invention by reference to specific embodiments. This exemplification, white illustrating certain specific aspects of the invention, does not portray the limitations or circumscribe the scope of the disclosed invention.

### 1. Example 1

Evaluation of the effects of compound IC on human lipid profiles

Sixty (60) patients comprised of male and non-fertile female patients aged at least 30 years with a diagnosis of Type 2 diabetes mellitus of at least three months duration, who have been treated with diet alone for at least one month prior to study entry were selected. The study was broken down into two periods. Period 1 was the four weeks prior to the beginning of the study, with period 2 being four weeks and being the actual study period when patients were treated with compound IC. Accordingly, study entry was Week −4 and the endpoint was after the fourth week of Period 2.

Patients were randomized in a ratio of 1:1:1 as follows: compound IC at 200 mg once a day (OD), compound IC at 100 mg OD and placebo. The patients received compound IC 30 minutes before breakfast. There were 5 test days in the study. Patients attended as outpatients for fasting blood sampling at Week −4 (study entry), Week −2 and Week 2 and as inpatients for 24 hours on Week 0 (=baseline) and Week 4 (=endpoint). On the two inpatient test days, the total caloric intake of breakfast, lunch and dinner was standardized and standard test meals were administered in place of breakfast and dinner. Triglycerides, total cholesterol and lipid fractions (LDL, VLDL and HDL) were measured during 24 hours following the breakfast standard meal.

On the three outpatient test days, patients fasted for at least 7 hours (i.e., no food or drinks (except water) after midnight on the day before the scheduled visit) and attended between 07.00 and 10.00 h and did not take the morning dose of compound IC.

On the two inpatient test days, patients fasted for at least 7 hours, i.e., no food or drinks (except water) after midnight on the day before the scheduled visit, and attended the clinic at 07.00 h. On each of the two test days, the total caloric intake during 24 hours was standardized and standard test meals were administered for breakfast (about 08.00 h) and dinner (about 18.00 h). Lunch was taken at approximately 13.00 h. On Day 1, no compound IC was administered but at Week 4, patients took compound IC as normal, 30 minutes before the standard breakfast. Triglycerides, total cholesterol and lipid fractions (LDL, VLDL and HDL) were evaluated. Triglycerides, total cholesterol and HDL were measured and LDL and VLDL calculated according to the method of Friedewald et al., “Estimation of the Concentration of Low-Density Lipoprotein Cholesterol Without the Use of the Preparative Centrifuge”, Clin. Chem., Vol. 18, No. 6, pp. 499-502 (1972).

Illustrative of the invention, compound IC markedly lowered levels of triglyceride, total cholesterol, LDL and VLDL compared to placebo.

Although the present invention has been described in considerable detail with reference to certain preferred versions thereof, other versions are possible without departing from the spirit and scope of the preferred versions contained herein. All references and patents (U.S. and others) referred to herein are hereby incorporated by reference in their entirety as if set forth in full herein.

1. A method of preventing and/or treating hyperlipidemia and or conditions associated with hyperlipidemia comprising administering to a mammal in need thereof a therapeutically effective amount of a compound of formula I: 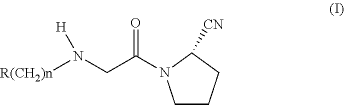
wherein
R is substituted adamantyl; and
N is 0 to 3; in free form or in acid addition salt form.

2. (canceled)

3. A pharmaceutical composition comprising a compound of formula I, or a pharmaceutically acceptable salt thereof and a pharmaceutically acceptable carrier.

4. A pharmaceutical composition comprising (a) a compound of formula I, and at least one compound selected from the group consisting of (b) an antihyperlipidemic agent; a plasma HDL-raising agent; an antihypercholesterolemic agent, such as a cholesterol biosynthesis inhibitor, e.g., an HMG-CoA reductase inhibitor, an HMG-CoA synthase inhibitor, a squalene epoxidase inhibitor or a squalene synthetase inhibitor; an ACAT inhibitor; probucol; nicotinic acid and the salts thereof and niacinamide; a cholesterol absorption inhibitor; a bile acid sequestrant anion exchange resin; an LDL receptor inducer; a cholesterol absorption inhibitor; fibrates; vitamin B6 and the pharmaceutically acceptable salts thereof; vitamin B12; vitamin B3; anti-oxidant vitamins; a β-blocker; an angiotensin II receptor (AT₁) antagonist; an angiotensin-converting enzyme inhibitor; a renin inhibitor, and a platelet aggregation inhibitor, a fibrinogen receptor antagonists, a glycoprotein IIb/IIIa fibrinogen receptor antagonists; and aspirin.

5. A method of claim 1, wherein the compound of formula I is a compound selected from a compound of formulae IA or IB: 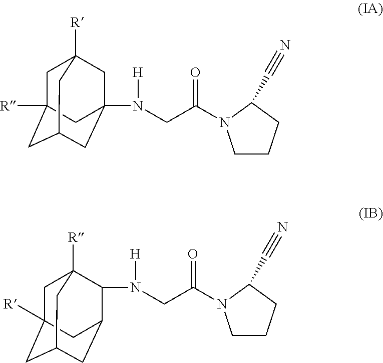
wherein R′ represents hydroxy, C₁-C₇alkoxy, C₁-C₈alkanoyloxy, or R₅ R₄ N—CO—O—, where R₄ and R₅ independently are C₁-C₇alkyl or phenyl which is unsubstituted or substituted by a substituent selected from C₁-C₇alkyl, C₁-C₇alkoxy, halogen and trifluoromethyl and where R₄ additionally is hydrogen; or R₄ and R₅ together represent C₃-C₆alkylene; and R″ represents hydrogen; or R′ and R″ independently represent C₁-C₇alkyl; in free form or in form of a pharmaceutically acceptable acid addition salt.

6. A method of claim 1, wherein the compound of formula I is a compound of formula IC.

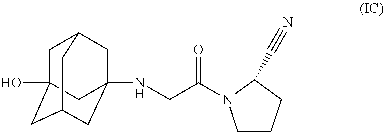

7. A method of claim 1, wherein the conditions associated with hyperlipidemia are selected from the group consisting of atherosclerosis, angina pectoris, carotid artery disease, cerebral arteriosclerosis, xanthoma, CHD, ischemic stroke, restenosis after angioplasty, peripheral vascular disease, intermittent claudication, reduction in necrosis after myocardial infarction, dyslipidemia, post-prandial lipemia.

8. A method of preventing and/or treating hyperlipidemia and/or conditions associated with hyperlipidemia comprising administering to a mammal in need thereof a therapeutically effective amount of a compound of formula I: 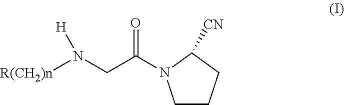
wherein
R is substituted adamantyl;
N is 0 to 3; in free form or in acid addition salt form; and
another active agent.

9. A method of lowering LDL, Lp(a) and/or VLDL levels in a mammal comprising administering to a mammal a therapeutically effective amount of a compound of formula I and another active agent.

10. (canceled)

11. The method of claim 8, wherein the compound of formula I is a compound of formula IC.

12. The method of claim 8, wherein the active agent is selected from the group consisting of an antihyperlipidemic agent; a plasma HDL-raising agent; an antihypercholesterolemic agent, such as a cholesterol biosynthesis inhibitor, e.g., an HMG-CoA reductase inhibitor, an HMG-CoA synthase inhibitor, a squalene epoxidase inhibitor or a squalene synthetase inhibitor; an ACAT inhibitor; probucol; nicotinic acid and the salts thereof and niacinamide; a cholesterol absorption inhibitor; a bile acid sequestrant anion exchange resin; an LDL receptor inducer; a cholesterol absorption inhibitor; fibrates; vitamin B6 and the pharmaceutically acceptable salts thereof; vitamin B12; vitamin B3; anti-oxidant vitamins; a β-blocker; an angiotensin II receptor (AT₁) antagonist; an angiotensin-converting enzyme inhibitor; a renin inhibitor, and a platelet aggregation inhibitor, a fibrinogen receptor antagonists, a glycoprotein IIb/IIIa fibrinogen receptor antagonists; and aspirin.

13. The method of claim 8, wherein the conditions associated with hyperlipidemia are selected from the group consisting of atherosclerosis, angina pectoris, carotid artery disease, cerebral arteriosclerosis, xanthoma, CHD, ischemic stroke, restenosis after angioplasty, peripheral vascular disease, intermittent claudication, reduction in necrosis after myocardial infarction, dyslipidemia, post-prandial lipemia.

14. The pharmaceutical composition of claim 4, wherein the active agent (b) is selected from the group consisting of, statins; bile acid-binding resins; nicotinic acid, probucol, β-carotene, vitamin E or vitamin C.

15. The pharmaceutical composition of claim 4, wherein the active agent (b) is selected from the group consisting of fluvastatin, lovastatin, pravastatin, atorvastatin or simvastatin.

16. The pharmaceutical composition of claim 4, wherein the compound of formula I is a compound of formula IC and wherein the active agent (b) is selected from the group consisting of fluvastatin, lovastatin, pravastatin, atorvastatin or simvastatin.

17. (canceled)

18. The method of claim 9, wherein the compound of formula I is a compound of formula IC.

19. The method of claim 9 wherein the active agent is selected from the group consisting of an antihyperlipidemic agent; a plasma HDL-raising agent; an antihypercholesterolemic agent, such as a cholesterol biosynthesis inhibitor, e.g., an HMG-CoA reductase inhibitor, an HMG-CoA synthase inhibitor, a squalene epoxidase inhibitor or a squalene synthetase inhibitor; an ACAT inhibitor; probucol; nicotinic acid and the salts thereof and niacinamide; a cholesterol absorption inhibitor; a bile acid sequestrant anion exchange resin; an LDL receptor inducer; a cholesterol absorption inhibitor; fibrates; vitamin B6 and the pharmaceutically acceptable salts thereof; vitamin B12; vitamin B3; anti-oxidant vitamins; a b-blocker; an angiotensin II receptor (AT1) antagonist; an angiotensin-converting enzyme inhibitor; a renin inhibitor, and a platelet aggregation inhibitor, a fibrinogen receptor antagonists, a glycoprotein IIb/IIIa fibrinogen receptor antagonists; and aspirin.

20. The method of claim 11 wherein the active agent is selected from the group consisting of an antihyperlipidemic agent; a plasma HDL-raising agent; an antihypercholesterolemic agent, such as a cholesterol biosynthesis inhibitor, e.g., an HMG-CoA reductase inhibitor, an HMG-CoA synthase inhibitor, a squalene epoxidase inhibitor or a squalene synthetase inhibitor; an ACAT inhibitor; probucol; nicotinic acid and the salts thereof and niacinamide; a cholesterol absorption inhibitor; a bile acid sequestrant anion exchange resin; an LDL receptor inducer; a cholesterol absorption inhibitor; fibrates; vitamin B6 and the pharmaceutically acceptable salts thereof; vitamin B12; vitamin B3; anti-oxidant vitamins; a b-blocker; an angiotensin II receptor (AT1) antagonist; an angiotensin-converting enzyme inhibitor; a renin inhibitor, and a platelet aggregation inhibitor, a fibrinogen receptor antagonists, a glycoprotein IIb/IIIa fibrinogen receptor antagonists; and aspirin.

21. The method of claim 8, wherein the active agent (b) is selected from the group consisting of, statins; bile acid-binding resins; nicotinic acid, probucol, b-carotene, vitamin E or vitamin C.

22. The method of claim 9, wherein the active agent (b) is selected from the group consisting of, statins; bile acid-binding resins; nicotinic acid, probucol, b-carotene, vitamin E or vitamin C.

23. The method of claim 8, wherein the active agent (b) is selected from the group consisting of fluvastatin, lovastatin, pravastatin, atorvastatin or simvastatin.

24. The method of claim 9, wherein the active agent (b) is selected from the group consisting of fluvastatin, lovastatin, pravastatin, atorvastatin or simvastatin.

25. The method of claim 8, wherein the compound of formula I is a compound of formula IC and wherein the active agent (b) is selected from the group consisting of fluvastatin, lovastatin, pravastatin, atorvastatin or simvastatin.

26. The method of claim 9, wherein the compound of formula I is a compound of formula IC and wherein the active agent (b) is selected from the group consisting of fluvastatin, lovastatin, pravastatin, atorvastatin or simvastatin.
